(19) United States
(12) Patent Application Publication
(10) Pub. No.: US 2011/0123572 A1
(43) Pub. Date: May 26, 2011

(54) ACTIVATED FIBROBLASTS FOR TREATING TISSUE AND/OR ORGAN DAMAGE

(75) Inventors: Jozef Bizik, Slovakia (SK); Ari Harjula, Helsinki (FI); Esko Kankuri, Helsinki (FI); Antti Vaheri, Helsinki (FI)

(73) Assignee: Licentia Ltd., Helsinki (FI)

(21) Appl. No.: 12/377,585

(22) PCT Filed: Aug. 16, 2007
(86) PCT No.: PCT/FI2007/050448

§ 371 Date: Mar. 18, 2009

(30) Foreign Application Priority Data
Aug. 16, 2006 (FI) 20065514

(51) Int. Cl. A61K 9/00; A61K 35/12; A61K 38/18; A61K 38/19; C12N 5/071; A61P 17/02; A61P 43/00; A61P 9/10; A61P 9/00

(52) U.S. Cl. 424/400; 424/572; 514/7.6; 514/9.5; 435/325

(57) ABSTRACT

The invention provides activated fibroblasts for the treatment of tissue and/or organ damage in a patient. Fibroblasts are activated by culturing the cells under conditions that induce the cells to adhere to each other and simultaneously to secrete growth factors, especially hepatocyte growth factor, HGF. The invention also provides a pharmaceutical composition based on the medium in which the activated fibroblasts are cultured. The invention further provides methods for transplantation.

The present invention relates to a field of tissue engineering which holds huge potential to treat damages in a wide range of tissues. The invention provides activated fibroblasts for the treatment of tissue and/or organ damage in a patient. Fibroblasts are activated by culturing the cells under conditions that induce the cells to adhere to each other and simultaneously to secrete growth factors, especially hepatocyte growth factor, HGF. The invention also provides a pharmaceutical composition based on the medium in which the activated fibroblasts are cultured. The invention further provides methods for transplantation.

### BACKGROUND OF THE INVENTION

The regeneration capacity of injured human tissues is extremely limited. Transplantation of the entire organ is ultimately required to replace the non-functional tissue. Recently, a novel concept of autologous cell transplantation for tissue regeneration therapy has emerged as an alternative and safe approach for treatment and prevention of tissue dysfunction and cell death (see, e.g., published patent applications US 2002/0197240 and US 2005/0025749). In this approach precursor or stem cells with variable capacity to differentiate to the preferred host tissue phenotype are harvested from a patient, enriched ex vivo, and transplanted to the same patient.

Heart failure and wound healing, are common, therapeutically highly challenging, and costly medical problems, the treatment of which would both highly benefit from induction of regeneration capacity of the tissue flanking the injured site. Heart failure has an overall prevalence of 3% in the adult population increasing with age so that every tenth over 65-year-old suffers from it. The one-year mortality rates range from 10-40%, and approximately 60% of patients die by five years from the time of diagnosis. Cell therapy has been administered safely to 200-300 patients, and despite promising results maximal efficacy will require novel approaches and their evaluation in randomized placebo-controlled clinical trials. The yearly incidence of burns requiring medical attention has been estimated to be 1.1 million in the United States and 12 000 in Finland. Of these 45 000, and 1 600, respectively, require hospitalization. Due to ineffective and time consuming therapeutic approaches many patients with severe burns die from wound infections, usually from pneumonia or from septic shock. Autologous skin grafting is widely used for re-epithelization of wounds. The grafts need to be extended (meshed) to cover large wound areas resulting frequently in improper attachment and defect coverage by the graft.

In order to stimulate the regeneration capacity of injured tissues, the site of damage should be in contact with natural growth factors. For this purpose, US 2005/0271697 discloses a method for local delivery of growth factors. In the method, a stent containing growth factors within openings in the stent is implanted to the site of transplantation therapy.

The present invention provides novel means for local delivery of growth factors by activating fibroblast cells prior to transplantation in vitro to produce growth factors and then transplanting the activated fibroblasts to the site of tissue damage.

As found in the invention, secretion of hepatocyte growth factor/scatter factor (HGF/SF) by the activated fibroblasts is maximally on the level of 15 ng of HGF/ml/10⁶ cells, which is 30-times more than can be achieved by adenoviral transfections or by systemic administration of recombinant HGF as reported in the literature (Jin et al., 2003; Li et al., 2003).

Bizik et al. recently published inaugural observations on a novel form of cell activation and phenotype switching in human dermal fibroblasts (Bizik et al., 2004). The authors showed that when mesenchymal cells are deprived of normal extracellular matrix contacts, they are forced to adhere to each other. Further, Kankuri et al. (2005) disclose induction of HGF secretion by fibroblast clustering but do not disclose any clinical use for the clustered fibroblasts.

### BRIEF DESCRIPTION OF THE DRAWINGS

FIGS. 1a-1c. Morphology, metabolic activity, and LDH release during the formation and decomposition of dermal fibroblast spheroids. Aliquots of 1_{—}104 cells were seeded to U-bottom wells treated with agarose to prevent cell spreading and cultured as described Example 1. (a) Phase-contrast microphotographs of the spheroids taken at the indicated time points (bars ¼ 300 mm). Cellular debris around spheroids is indicative of decomposition after 48 h of incubation. (b) Cells were grown concurrently as three-dimensional spheroids (-′-) or monolayers (—K—). Metabolic activity measured by MTT assay. Substrate MTT was added to cultures at different time points and incubated for 30 min. Substrate conversion to formazan was estimated by its optical density at 590 nm. (c) Membrane leakage was measured by LDH activity in conditioned medium harvested at indicated time points after addition of specific substrate. Optical density was measured and values expressed as percentage of total initial load. Data are means of triplicate cultures [symbol] S.E.M. *Po0.001 between cultures

FIGS. 2a and 2b. (a), Production of hepatocyte growth factor (HGF/SF) by spheroids and corresponding monolayer cultures. Concentrations of HGF/SF were measured from conditioned medium of cells cultured concurrently as three-dimensional spheroids (▪) or monolayers (▴). ** p<0.001 as compared to corresponding monolayers. Data are mean±SEM from three independent experiments. (b), analysis of HGF/SF molecular weight species as produced by spheroids and monolayers of fibroblasts. Three different molecular weight species of HGF/SF were detectable in immunoblot analysis of conditioned medium from spheroid cultures. Arrows, 180-kDa mature HGF/SF, and the HGF/SF heavy and light chains at 60 and 30 kDa, respectively. The corresponding conditioned medium from monolayer cultures was negative for HGF/SF by immunoblotting. Conditioned medium from both spheroid and monolayer cultures of fibroblasts was collected after 96 hours of culture.

FIG. 3. Induction Qf COX-2 in fibroblast spheroids cultured for indicated times. No induction of the constitutive COX-1 iso form was evident.

FIG. 4. Expression of COX-2 is induced and uniform within fibroblast spheroids by immunohistochemistry.

FIG. 5. Production of pro-angiogenic prostaglandins PGE₂ and PGI₂ is induced in fibroblast spheroids. Concentrations determined by ELISA from conditioned medium.

FIG. 6. Stimulation of HMEC-1 endothelial cells with nemosis-derived factors leads to endothelial cells tubule formation suggesting high pro-angiogenic activity by prostaglandins and HGF/SF from fibroblasts in nemosis.

FIG. 7. Phase-contrast microphotographs showing the spheroid formation due to nemosis of bone marrow stromal cells.

FIG. 8. Production of HGF in cultured bone marrow stromal cells.

FIG. 9. Wound healing stimulated by treating with bone marrow stromal cell (BMSC) spheroids: right: 0 d, left: 3 d.

FIG. 10. Keratinocytes were stimulated with indicated amounts of mesenchymal stem cell spheroids or their respective amounts of mesenchymal stem cells grown as standard monolayer cultures. A clear dose-response on wound healing is seen in only the nemotic cells, whereas monolayer cultures show a minor effect that has no dose response, suggesting a nonspecific effect of cells cultured as monolayers on wound healing, and a specific effect of factors (that is at least HGF) released from spheroid, nemotic, structures.

### DETAILED DESCRIPTION OF THE INVENTION

Dermal fibroblasts from skin biopsies present the most lucrative and easily accessible superficial cell source for autologous transplantation therapy. They have a high proliferation capacity, but lack the contractile properties of muscle cells. However, in the present invention we have found that activated fibroblasts produce massive local concentrations of hepatocyte growth factor, HGF, a most powerful mitogen, motogen, and inducer of angiogenesis.

In this invention, the fibroblast cells are deprived of normal extracellular matrix contacts and are forced to adhere to each other. This leads to an activation of surface receptors and intracellular signaling in 3D culture. Compact spheroids can be formed from single cell suspensions of autologous fibroblasts within 12 h, and they reach their final tight form by 24 h.

In heart failure, in vitro induction of phenotype switch in isolated autologous skin fibroblast (or mesenchymal stem cell) spheroids, prior to transplantation, presents a new means for administering growth factor therapy, inducing therapeutic angiogenesis, and promoting growth and inhibiting apoptosis of resident and of co-transplanted cells. Moreover, we have shown that these activated fibroblast cells may die in the process, and thus do not populate the myocardium. This is a novel approach developed by the present inventors, and has the potential in the future to replace the need for viral transduction in gene therapy and cell transplantation. It needs to be stressed that no extrinsic stimuli or cell manipulation have to be used in the process.

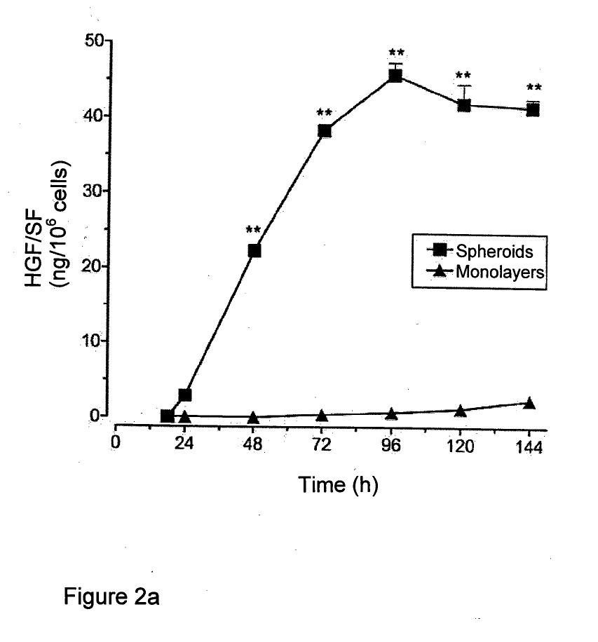

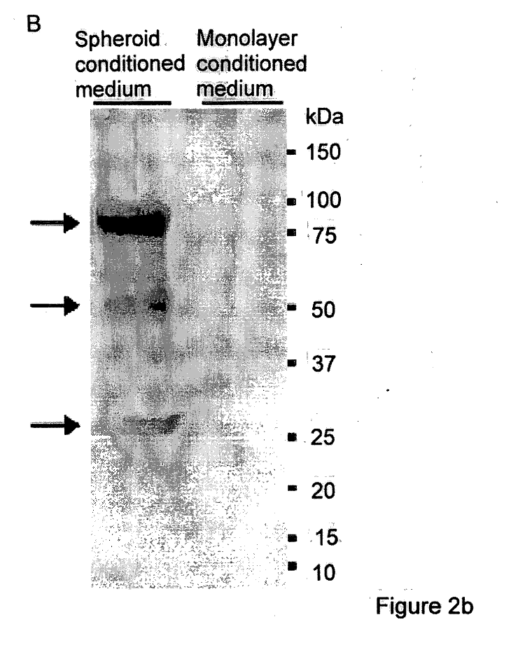

We have performed oligonucleotide microarray analysis (Affymetrix Inc.) of the fibroblast spheroids. This genome-wide array detected a 1000-fold upregulation of HGF messenger-RNA in the spheroids undergoing nemosis. As found in the microarrays, induction of HGF production was verified on protein level, by estimation of secreted HGF in culture medium of activated fibroblasts (FIG. 2A). Moreover, the produced HGF was found to be secreted in a cleaved and bioactive form. Secretion of maximally 15 ng/ml/10⁶ cells of HGF is 30-times more than can be achieved by adenoviral transfections or by systemic administration of recombinant HGF as reported in the literature (Jin et al., 2003; Li et al., 2003).

Skin injuries extending to the dermis and beyond require surgical intervention and usually the area of the defect must be covered either by skin transplantation or by an artificial skin replacement after evacuation of any underlying damaged tissues. The ideal covering is a meshed skin autograft taken from the unburned area. When using autologous cells the graft is not rejected and is accepted as a permanent patch by the body's immune system. In patients with severe burns the lack of available donor sites limits autologous transplantation requiring extensive graft meshing and stretching resulting in poor cell outgrowth and graft integration into the host site. The present invention provides novel means to promote autologous skin graft-based therapy, graft persistence, and wound healing using activated fibroblast-based therapy approach.

Other tissues treatable by the invention are, e.g., brain tissue, bone tissue, connective tissue, cartilage, and muscular tissue.

The invention provides a method for treating tissue and/or organ damage in a patient. The method comprises the following steps:

a) culturing fibroblast cells under conditions that induce the cells to adhere to each other so that multicellular spheroids are formed;
b) transplanting said multicellular spheroids obtained from step a) to a site of tissue or organ damage.

The invention is particularly directed to the treatment of ischemic damage in the heart, when the damaged site is at myocardium. Grafting of cells into the myocardium requires some form of delivery system. The choice for the routes of cell implantation may depend on the pathology of the heart. Up to now, most of studies in the field of cellular cardiomyoplasty were performed by direct injection of various cells into the myocardium (e.g., Connold et al., 1997; Soonpaa et al., 1994; and Koh et al., 1993). Thus, multicellular fibroblast spheroids, i.e. activated fibroblasts, obtained from step a) are preferably injected to the site of damage in the heart tissue. The cells can be administered in a physiologically compatible carrier, such as a buffered saline solution.

Multicellular fibroblast spheroids can also be transplanted by topical administration, especially when the tissue damage is a skin burn or a skin wound. Said topical administration may be performed by the use of an adhesive bandage, wherein said bandage comprises said multicellular fibroblast spheroids.

Fibroblast cells for the culture in step a) are preferably obtained from skin biopsy. However, any kind of fibroblasts capable of forming multicellular fibroblast spheroids can be used in the invention. Such fibroblasts are, e.g., stromal fibroblasts, bone marrow stromal cells and mesenchymal stem cells.

The safest approach for transplantation is to use patient's own cells for the transplant, therefore the preferred embodiment of the invention is to use autologous fibroblasts for the culture in step a). However, also allogeneic fibroblasts can be used in the invention, since the activated fibroblast cells die after the transplantation, and thus do not populate the transplantation site and cause host-graft rejection.

Typically, multicellular fibroblast spheroids are transplanted together with another type of cells to a site of tissue or organ damage. These other types of cells are preferably stem cells, e.g., selected from the group consisting of: bone marrow stromal cells, hematopoietic cells, brain stem cells, neural stem cells, cardiomyoblasts, or other endogenous stem cells.

Another embodiment of the invention is a method for treating tissue and/or organ damage in a patient. This method comprises the following steps:

a) culturing fibroblast cells under conditions that induce the cells to adhere to each other so that multicellular spheroids are formed;
b) incubating said multicellular sphreoids under conditions that said fibroblast cells secrete growth factors to the culture medium;
c) separating the cells from the culture medium;
d) administering a solution comprising culture medium obtained from step c) to a site of tissue or organ damage.

Preferably, one of the secreted growth factors in step b) is hepatocyte growth factor, HGF.

Typically, the solution in step d) comprises a purified fraction of the culture medium obtained from step c). The purification of the culture medium can be performed in various ways well-known to a skilled artisan to yield a purified solution consisting of one type of molecules or a mix of several types of molecules.

Preferably, the solution comprising culture medium obtained from step c) is injected to the site of tissue or organ damage. Injection is particularly preferable method for administration, when the ischemic damage in the heart is treated and the site of tissue or organ damage is myocardium. The solution comprising culture medium obtained from step c) can also be administered topically, especially when the tissue damage is a skin burn or a skin wound. Topical administration may also be performed by the use of an adhesive bandage, wherein said adhesive bandage comprises said culture medium or a lyophilisate or concentrate thereof.

Fibroblast cells for the culture in step a) are preferably obtained from skin biopsy. Since no cells are administered to a patient in this method, the use of allogeneic fibroblast in the culture should cause no problem to the patient to be treated.

The solution comprising culture medium obtained from step c) can also be administered to a site of tissue or organ damage simultaneously with a transplant. The transplant may contain stem cells as listed above.

One object of the invention is to provide pharmaceutical composition for use in treating tissue and/or organ damage in a patient. The pharmaceutical composition of the invention comprises multicellular spheroids obtainable by culturing fibroblast cells under conditions that induce the cells to adhere to each other so that multicellular spheroids are formed.

Preferably, the pharmaceutical composition of the invention is a transplant or graft to be transplanted to a patient. The transplant may be in a form of an injectable or topical solution.

One further object of the invention is a use of multicellular spheroids obtainable by culturing fibroblast cells under conditions that induce the cells to adhere to each other so that multicellular spheroids are formed, for the manufacture of a composition, such as a transplant or graft, for use in treating tissue and/or organ damage in a patient.

Another object of the invention is to provide a pharmaceutical composition comprising a solution obtainable by culturing fibroblast cells under conditions that induce the cells to adhere to each other so that multicellular spheroids are formed, incubating said multicellular sphreoids under conditions that said fibroblast cells secrete growth factors to the culture medium, and separating the cells from the medium. The separation of cells can be performed in various ways well-known to a skilled artisan to yield a purified solution consisting of one type of growth factors or a mix of several types of growth factors. This composition is also used for treating tissue and/or organ damage in a patient, preferably as an injectable or topical solution.

Another object of the invention is a use of a solution obtainable by culturing fibroblast cells under conditions that induce the cells to adhere to each other so that multicellular spheroids are formed, incubating said multicellular sphreoids under conditions that said fibroblast cells secrete growth factors to the culture medium, and separating the cells from the medium, for the manufacture of a composition, such as injectable or topical solution, for use in treating tissue and/or organ damage in a patient.

In above embodiments of the invention, the activated fibroblast cells can be suspended in a solution or embedded in a support matrix when used as a transplant or graft. Preferably, the solution includes a pharmaceutically acceptable carrier or diluent in which the cells of the invention remain viable. Pharmaceutically acceptable carriers and diluents include saline, aqueous buffer solutions, solvents and/or dispersion media. The use of such carriers and diluents is well known in the art. The solution is preferably sterile and fluid. Preferably, the solution is stable under the conditions of manufacture and storage and preserved against the contaminating action of microorganisms such as bacteria and fungi through the use of, for example, parabens, chlorobutanol, phenol, ascorbic acid, or thimerosal. Solutions of the invention can be prepared by incorporating the cells as described herein in a pharmaceutically acceptable carrier or diluent and, as required, other ingredients.

Support matrices in which the cells of the invention can be incorporated or embedded include matrices which are recipient-compatible and which degrade into products that are not harmful to the recipient. Natural and/or synthetic biodegradable matrices are examples of such matrices. Natural biodegradable matrices include, for example, collagen matrices and alginate beads. Synthetic biodegradable matrices include synthetic polymers such as polyanhydrides, polyorthoesters, and polylactic acid. These matrices provide support and protection for the cells in vivo.

Finally, the present invention provides a method for producing biologically active hepatocyte growth factor, HGF, said method comprising:

a) culturing fibroblast cells under conditions that induce the cells to adhere to each other so that multicellular spheroids are formed;
b) incubating said multicellular sphreoids under conditions that said fibroblast cells secrete hepatocyte growth factor to the culture medium;
c) isolating or purifying hepatocyte growth factor from the culture medium.

The methods for culturing and incubating fibroblast cells and isolating proteins from the culture medium are well known to a skilled artisan.

All publications and patent applications cited in this specification are herein incorporated by reference as if each individual publication or patent application were specifically and individually indicated to be incorporated by reference. The following examples will assist those skilled in the art to better understand the invention and its principles and advantages. It is intended that these examples be illustrative of the invention and not limit the scope thereof.

### EXPERIMENTAL SECTION

### Example 1

### Materials and Methods

### Cell Cultures

Human dermal fibroblasts (HFSF#132) established from neonatal foreskin50 were kindly provided by Dr. Magdalena Eisinger, Memorial Sloan-Kettering Cancer Center, New York, N.Y., USA. These cells were cultured in DMEM/F-12 (1:1) medium (Gibco, Paisley, Scotland) supplemented with 10% fetal bovine serum (Gibco), 100 mg/ml streptomycin, and 100 U/ml penicillin and were used at passages 5-15. All the experiments were performed with cells cultured in the medium containing 1.0 mM Ca2 [symbol] , unless a different concentration is indicated. For the set of experiments on the calcium dependence of necrosis, CaCl2 was added to a low-calcium medium to reach the desired concentrations.

### Initiation and Growth of Spheroids

U-bottom 96-well plates (Costar, Cambridge, Mass., USA) were treated with 0.8% LE agarose (BioWhittaker, Rockland, Me., USA) prepared in sterile water to form a thin film of a nonadhesive surface. Fibroblasts were detached from culture dishes by trypsin/EDTA, and a single cellsuspension (4_{—}104 cells/ml) was prepared in a complete culture medium. To initiate spheroid formation, 250 ml aliquots were seeded into individual wells and the dishes incubated at [symbol] 371 C in 5% CO2 atmosphere.

To initiate the spheroid formation of bone marrow stromal cells the same method as disclosed herein was used.

### Electron Microscopy

Multicellular clusters were collected at 12 h time intervals, and fixed by immersion in 0.5% glutaraldehyde and 0.1M phosphate buffer at pH 7.2 for 2 h at [symbol] 41 C. The specimens were washed in 0.1M phosphate buffer at pH 7.2 and then postfixed by 1% OsO4, 0.1M sucrose and 0.1M phosphate buffer at pH 7.2 for 1 h at [symbol] 41 C. After fixation, the specimens were dehydrated in a graded series of ethanol, washed in propylene oxide, and embedded in Epon 812 (Serva, Heidelberg, Germany). Thin sections were cut with a Reichert OmU3 ultramicrotome (Reichert Jung, Vienna, Austria) and poststained with uranyl acetate and lead citrate. Electron micrographs were taken with a transmission electron microscope JEOL 1200EX (JEOL, Tokyo, Japan) operated at 60 kV. Cells (1.2_{—}105) were lysed using 50 ml of lysis buffer and then incubated for 20 h with a caspase-3 substrate, Z-DEVD-AMC, which upon cleavage by caspase-3 generates fluorescent products. Reactions were carried out at room temperature, and fluorescence was measured in a fluorescent plate reader using an excitation wavelength of 355 nm and an emission wavelength of 430 nm. Monolayers stimulated with camptothecin (13 mM) (Sigma) for 12 h were used as positive controls.

### Results

### Clustering of Dermal Fibroblasts

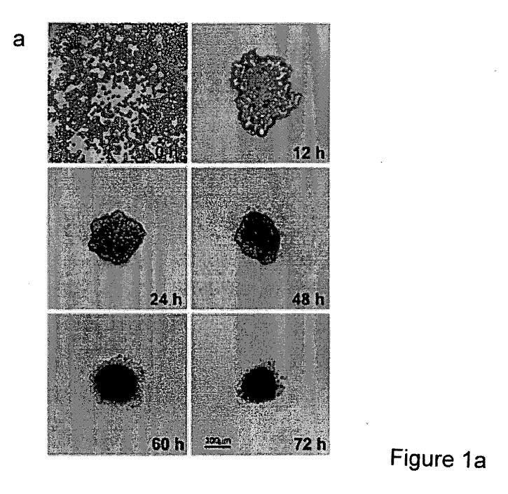

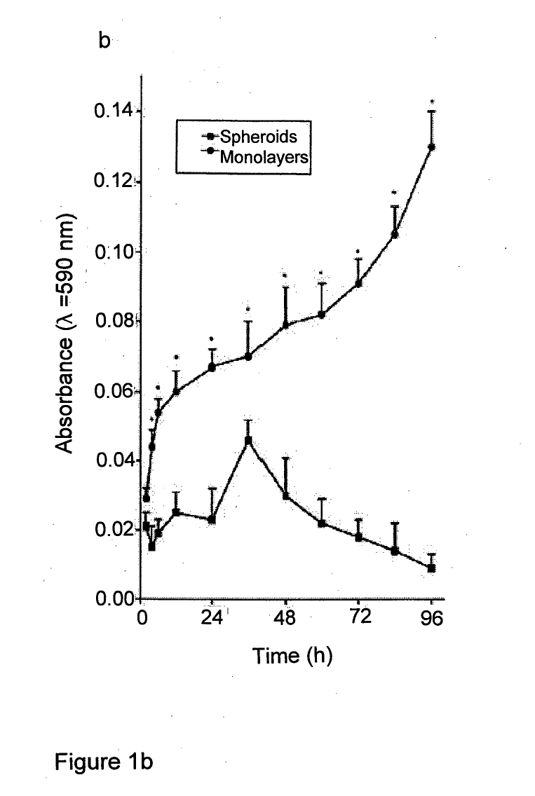

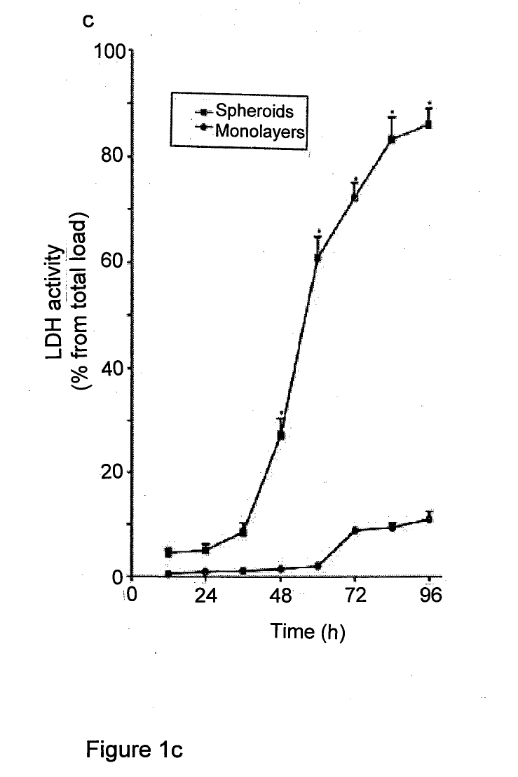

Prevention of fibroblast spreading on surfaces not favourable for attachment led to enhancement of homotypic cell-cell interactions, resulting in formation of multicellular clusters (FIG. 1a). In our study, cluster formation was initiated in Ubottom 96-well plates treated with agarose, which formed a thin nonadherent film. Agarose represents one of the most efficient substrates to prevent cell attachment,23 but poly(2-hydroxyethylmethacrylate) (polyHEMA), a nonionic compound which also prevents matrix deposition and subsequent cell attachment, worked equally well (data not shown). After the seeding of fibroblasts into the wells, they started to cluster, and the first loose aggregates were already seen after 2 h of incubation. Compact spheroids formed within 12 h, reaching their final tight form by 24 h (FIG. 1a). Once these aggregates were formed, the fibroblasts were so firmly attached to each other that the spheroids resisted disintegration by enzymatic treatment without showing substantial cell damage. The spheroids did not change in morphology during the next 24 h but then started gradually to decompose. This was visible as shedding of solitary shrunken and damaged cells and cellular fragments. The spheroid volume decreased as the degradation process continued, and after 120 h of incubation they were decomposed leaving remnants of dead cells, cellular fragments, and debris.

To assess cellular viability and metabolic activity of fibroblasts within the clusters, we applied MTT to the spheroid cultures and simultaneously to cultures of identical cell aliquots cultured as monolayers (FIG. 1b). Two-dimensional cultures of fibroblasts grown on standard adherent plastic surfaces exhibited characteristic exponential growth, whereas the cells in spheroids showed quantitatively a different growth pattern: their metabolic activity was practically at a residual level when compared to that of monolayers, and gradually declined. In spheroids, however, some increase in metabolic activity was evident at 36 h.

Conditioned media from these two types of cultures were also used for estimation of lactate dehydrogenase (LDH) release, which is considered a criterion for loss of membrane integrity.25 Concentration of LDH in the cultures with spheroids began to increase after 48 h of incubation, reaching a submaximal level at 84 h (FIG. 1c). The amounts of LDH released from monolayer cultures were at a marginal level, but with prolonged incubation, after 72 h some increase occurred.

### Ultrastructure of Decomposing Spheroids

Transmission electron microscopy demonstrated morphological changes in cells within the spheroids. Nuclei retained their typical morphology for up to 36 h, but thereafter, some aggregation of chromatin into ill-defined masses was evident. This pattern was most distinct at 60 h, but the integrity of nuclear membranes was still maintained. Later, the nuclei become pyknotic, and terminal spheroids showed mostly nuclear remnants. Marginal clumping of loosely textured nuclear chromatin was linked to early abnormalities, compatible with progression of necrosis. In spheroids after 48 h of incubation, phagolysosome-like dense bodies were numerous but patchy in their distribution; later they grew in size. Evidently, the spheroids underwent their most dramatic change in subcellular morphology after 48 h, since at 60 h balloon-like and autophagic vacuoles were present throughout, and as the spheroid degradation proceeded these were the dominant structures within the cell bodies. Ultrastructural features appearing during the spheroid decomposition process were characteristic of necrosis.

### Expression of Apoptosis-Related Genes in Spheroids

Fragmentation of chromatin is one of the hallmarks of apoptotic cell death. Our analysis of the genome integrity of DNA isolated from the spheroids by agarose gel electrophoresis revealed no fragments suggestive of interchromosomal DNA cleavage, even in highly decomposed spheroids, for example after 96 h of incubation (data not shown). We also performed terminal deoxynucleotidyltransferase-mediated dUTP nick-end labelling (TUNEL) staining of sectioned spheroids. In spheroids until 48 h of incubation less than 1% of cells were positive for DNA breaks, whereas after 96 h the majority were positive. As an indicator of necrosis, distinct TUNEL positivity occurred only during later stages of decomposition.

As most cells undergoing apoptosis are dependent on specific gene transcription and consequent de novo protein synthesis,4 we analysed the expression of some proteins involved in the apoptotic pathway: Bax, p53, the cell-surface receptor Fas, and the death-associated protein Daxx. All these proteins showed their highest expression in the early spheroids, but it declined as the spheroids decomposed. There was no evidence of upregulation of the antiapoptotic Bcl-2 protein. No active caspase-3, which plays a crucial role in the execution of the apoptotic process, was detected. Procaspase-3 expression had a similar pattern to that of the other components of apoptosis studied. The activity of caspase-3 was further evaluated with the aid of a specific substrate, Z-DEVD-AMC. No induction of caspase-3 activity was seen in the spheroids as compared to corresponding control monolayer cultures (data not shown). The apoptosis inducer camptothecin markedly induced caspase-3 activity (data not shown).

Heat shock proteins confer cellular protection against a variety of cytotoxic stress conditions and also against physiological stress associated with growth arrest or receptor-mediated apoptosis. Hsp27 and Hsp70 in particular may directly affect the execution of apoptotic signalling pathways by their capability to increase survival of cells exposed to a variety of lethal stimuli. We therefore analysed expression of these two proteins as well as that of calreticulin, an endoplasmic reticulum-specific chaperone, which facilitates induction of apoptosis. A similar decline in band intensity occurred as for apoptosis-related genes.

### Example 2

### Hepatocyte Growth Factor Abundantly Produced by Multicellular Fibroblast Spheroid Clusters

Similar to induction by stimulation in tumor cell-conditioned medium, fibroblast spheroids can be effectively initiated as described in Example 1. Conditioned medium from cultures of human fibroblast spheroids and corresponding monolayers was collected and quantified for HGF/SF by ELISA. Production of HGF/SF by fibroblast spheroids increased 220-fold at 48 hours compared with that detected in corresponding monolayer cultures (FIG. 2A). As shown in FIG. 2A, clustered fibroblasts produced up to 45 ng HGF/SF per 106 cells at 96 hours. An aliquot of the conditioned medium was separated by PAGE, and after immunoblotting, probed by specific anti-HGF/SF antibodies. Three bands with apparent Mr 80, 56, and 34 kDa were detectable in the spheroid-conditioned medium (FIG. 2B). As estimated by densitometric scanning of the immunoblot, the 80-kDa band represented 49%, the 56-kDa band represented 26%, and the 34-kDa band represented 25%. This pattern indicates that at least half the HGF/SF produced was in its active processed form. None of the tumor cell lines selected for the study produced HGF/SF, and values measured in their conditioned medium were at the background level of ELISA (data not shown).

### Example 3

Fibroblasts are the dominant cell type in mesenchymal tissues producing extra cellular matrix components and providing context and support for epithelial cells in tissues and organs. Their ubiquitous presence within the body makes them easy to isolate and cultivate. The role of fibroblasts in cell transplantation therapy for ischemic heart disease is generally underscored because of their association with fibrosis and scarring.

### Aim

We demonstrate here inaugural rationale for the use of fibroblasts in cell therapy for ischemic heart disease. We aimed to show that the recently identified novel fibroblast activation, nemosis (Bizik et al., 2004; Kankuri et al., 2005), produces angiogenic factors that promote experimental angio genesis. The results will provide a novel unprecedented role for the fibroblasts as cell therapy for patients with ischemic heart disease.

### Methods

Diploid human dermal fibroblasts were cultured on agarose-coated U-bottomed 96-well plates. FIG. 1 shows the progression of spheroid formation. Corresponding control monolayer cultures and human microvascular endothelial cells (HMEC-1) were grown on standard culture dishes. For the assays the cells were treated nemosis-derived conditioned medium (nemosis-CM) or with respective monolayer-derived control-CM.

Tubulogenesis was assessed by morphology, and proliferation by cell counting. A standardized wound-healing assay was used to assess endothelial cell migration from the wound borders.

### Results

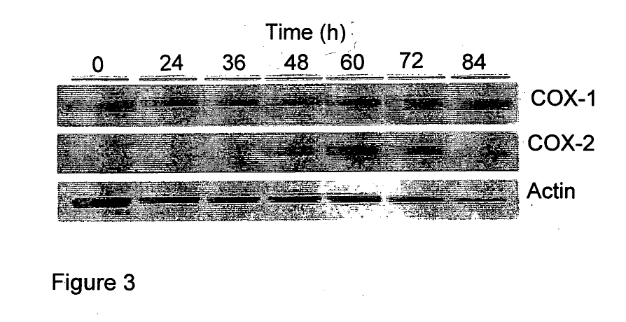

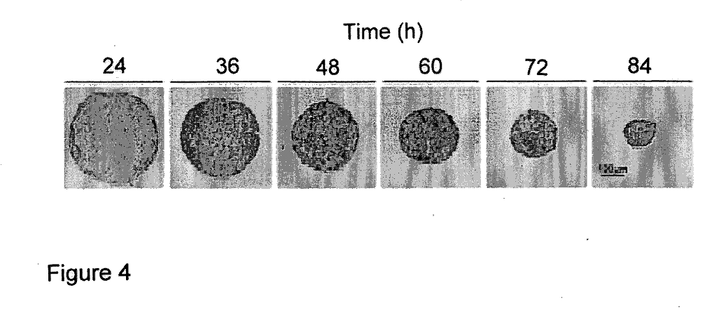

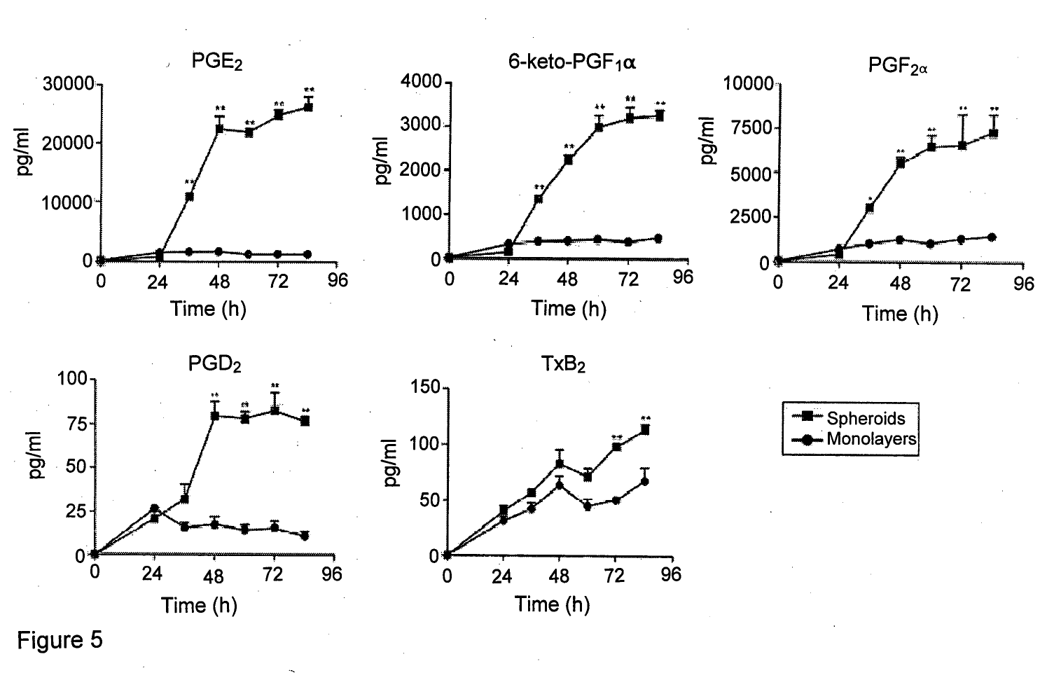

We found induction of the pro-angiogenic prostaglandin-producing cyclooxygenase-2 (COX-2) as shown in FIG. 3. FIG. 4 demonstrates uniform expression of COX-2 throughout the spheroid. COX-2 activity was reflected by an increased production of prostaglandins to the culture medium (FIG. 5). The induction of COX-2 expression and activity was accompanied by release of another potent pro-angiogenic factor, namely hepatocyte growth factor/scatter factor (HGF/SF) (FIG. 2a).

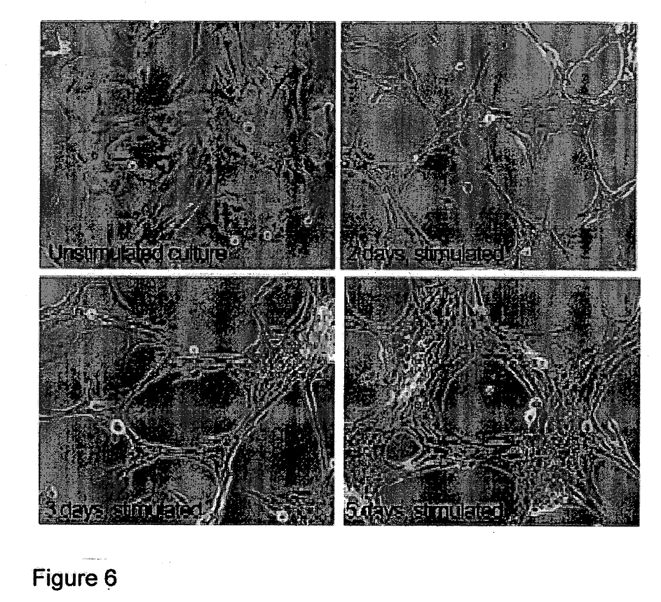

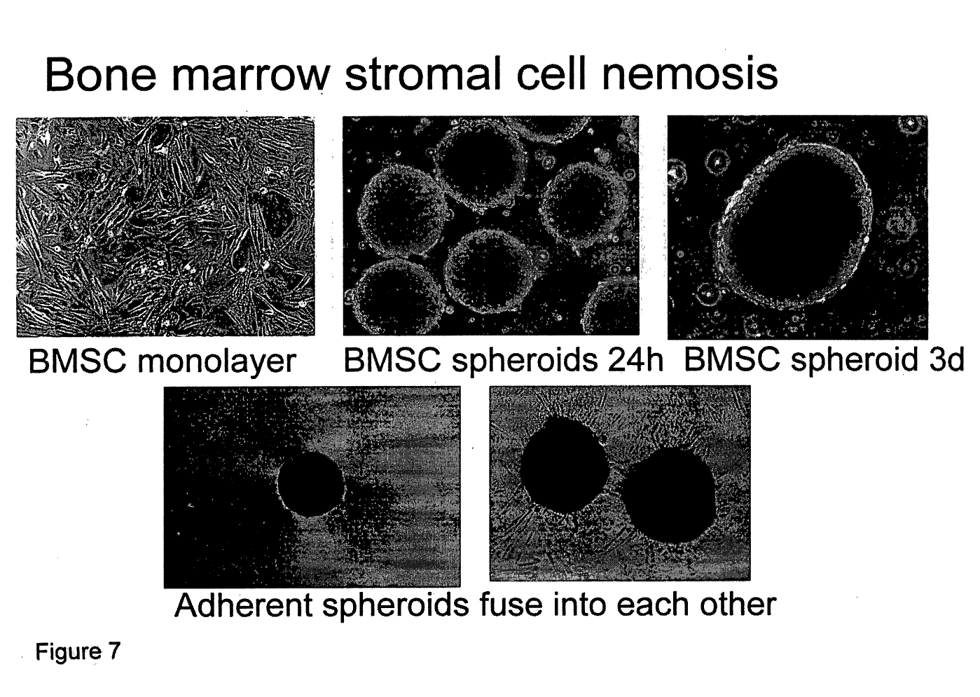

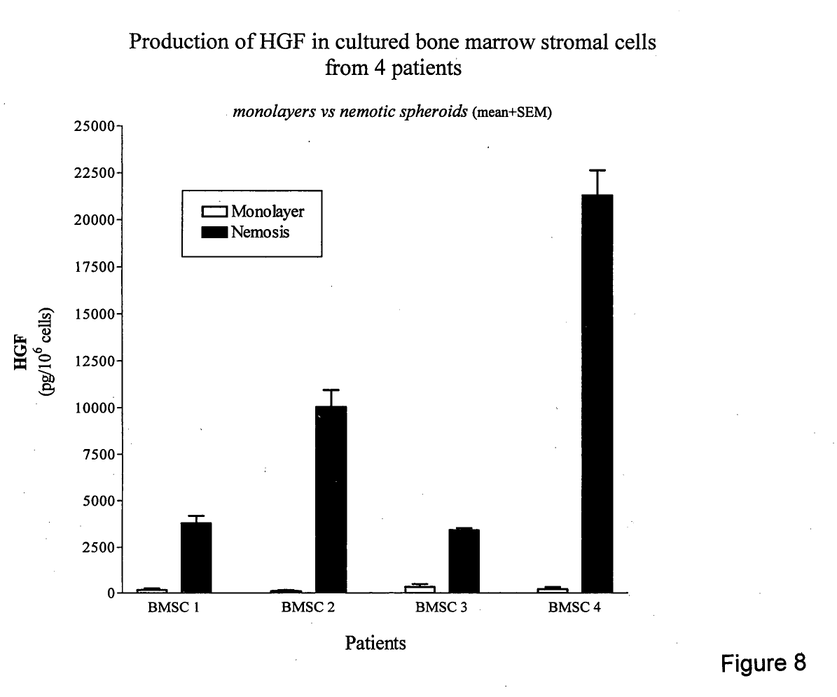

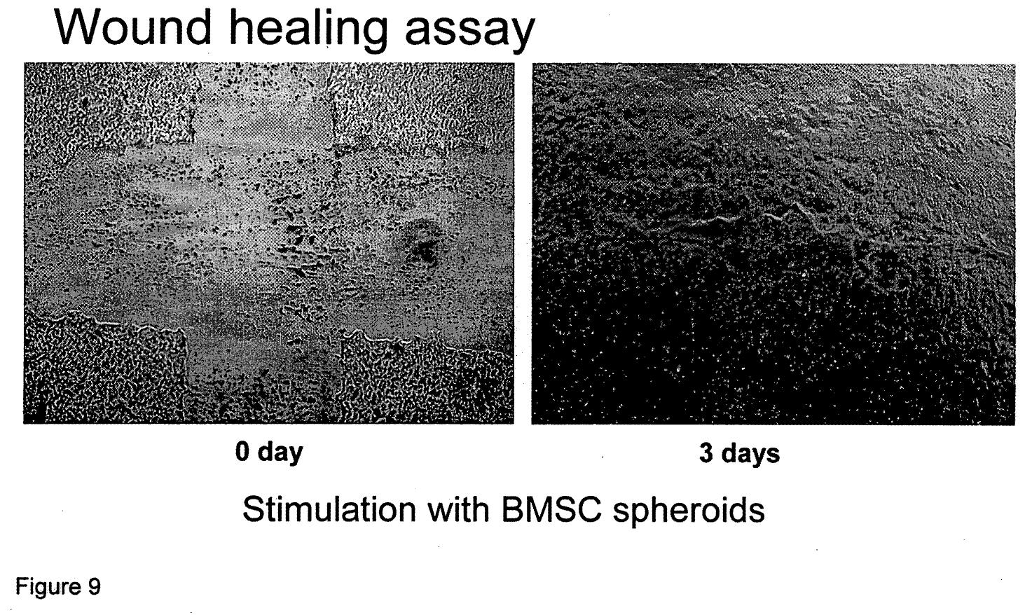

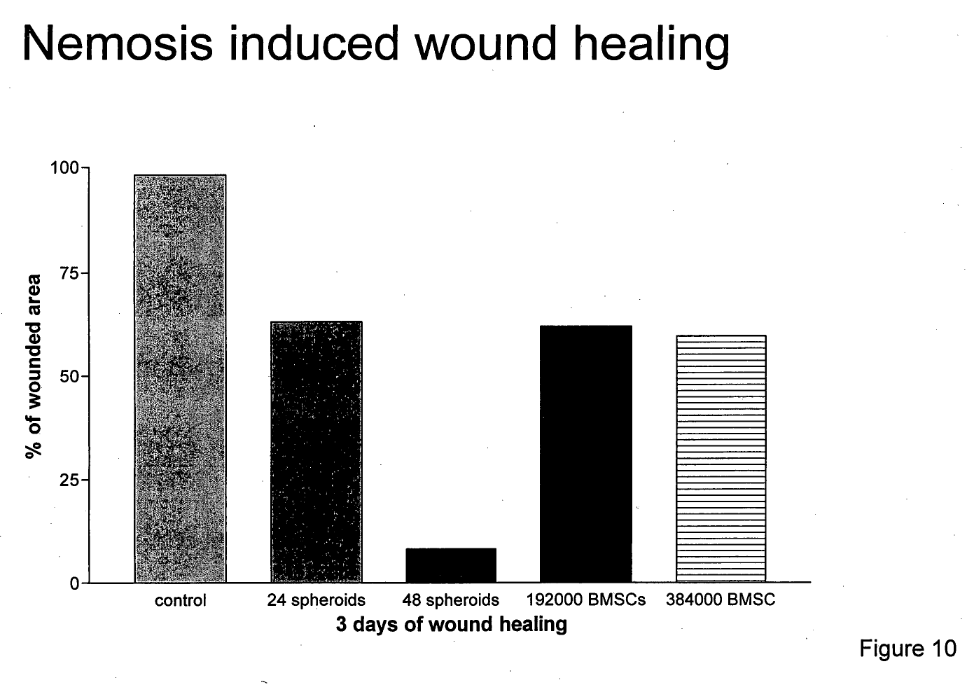

Fibroblasts in nemosis induced significant endothelial cell tubulogenesis at 48 hours after stimulation, whereas in control cells no tubulus formation was observed (FIG. 6). Nemosis-CM also stimulated endothelial cell proliferation 8.9-fold as compared to control-CM at 24 to 40 hours of incubation (nemosis-CM: 29.25±1.2-33.79±0.60*10⁴ cells vs. control-CM:25.31±0.63-25.82±1.09*10⁴ cells, p<0.05 at 24 hours, p<0.001 at 40 hours). Nemosis-CM induced a 15-fold increase in endothelial cell migration in the wound healing assay as compared to control-CM 23.33±5.58 vs. 1.50±0.27 cells/field, p<0.001).

### CONCLUSIONS

The novel fibroblast activation, nemosis, induces endothelial cell proliferation and tubulogenesis, and enhances migration rate in an experimental model of wound healing. We propose this effect of nemosis to be due to massive production of prostaglandins and active HGF/SF from the fibroblast spheroids.

These results present first rationale to the use of the novel fibroblast reactivity, nemosis, in cell transplantation therapy. We show that fibroblast nemosis is an ample source of factors stimulating angiogenesis in ischemic heart disease.

### REFERENCES

Bizik J, Kankuri E, Ristimaki A, Taieb A, Vapaatalo H, Lubitz W, Vaheri A. Cell-cell contacts trigger programmed necrosis and induce cyclooxygenase-2 expression. Cell Death Differ 11:183-195, 2004.
Connold A. L., Frischknecht R, Dimitrakos M, Vrbova G. The survival of embryonic cardiomyocytes transplanted into damaged host rat myocardium. J Muscle Res Cell Motil 1997; 18:63-70
Jin H et al. Early treatment with hepatocyte growth factor improves cardiac function in experimental heart failure induced by myocardial infarction. J Pharmacol Exp Ther 304:654-660, 2003.
Kankuri E, Cholujova D, Comajova M, Vaheri A, and Bizik J. Induction of hepatocyte growth factor/scatter factor by fibroblast clustering directly promotes tumor cell invasiveness. Cancer Research 65:9914-9922, 2005
Koh G Y, Klug M G, Soonpaa M H et al. Long-term survival of AT-1 cardiomyocyte grafts in syngenic myocardium. Am J Physiol 1993; 264: H-1727-H-1733.
Li Y et al. Postinfarction treatment with an adenoviral vector expressing hepatocyte growth factor relieves chronic left ventricular remodeling and dysfunction in mice. Circulation 107:2499-2506, 2003.
Soonpaa M H, Koh G Y, Klug M G, Field L J. Formation of nascent intercalated disks between grafted fetal cardiomyocytes and host myocardium. Science 264:98-101, 1994.

1-37. (canceled)

38. A method for treating tissue and/or organ damage in a patient, said method comprising:
a) culturing fibroblast cells on a nonadhesive surface that induces the cells to adhere to each other so that multicellular spheroids are formed and triggers necrosis of the cells;
b) transplanting said multicellular spheroids obtained from step a) to a site of tissue or organ damage.

39. The method according to claim 38, wherein the metabolic activity of the cells in multicellular spheroids during culturing is at a residual level when compared to that of monolayers, and then gradually declines.

40. The method according to claim 38, wherein said multicellular spheroids obtained from step a) are injected to the site of tissue or organ damage.

41. The method according to claim 38, wherein said organ damage is ischemic damage in the heart.

42. The method according to claim 38, wherein said site of tissue or organ damage is myocardium.

43. The method according to claim 38, wherein said multicellular spheroids obtained from step a) are transplanted by topical administration to a site of tissue damage.

44. The method according to claim 38, wherein said tissue damage is a skin burn or wound.

45. The method according to claim 38, wherein said fibroblast cells are autologous to the patient being treated.

46. The method according to claim 38, wherein said fibroblast cells are allogeneic to the patient being treated.

47. The method according to claim 38, wherein said fibroblast cells are obtained from skin biopsy.

48. The method according to claim 38, wherein in step b) said multicellular spheroids are transplanted together with another type of cells to a site of tissue or organ damage.

49. The method according to claim 38, wherein said fibroblast cells are bone marrow stromal cells.

50. A method for treating tissue and/or organ damage in a patient, said method comprising:
a) culturing fibroblast cells under conditions that induce the cells to adhere to each other so that multicellular spheroids are formed;
b) incubating said multicellular spheroids under conditions that said fibroblast cells secrete growth factors to the culture medium;
c) separating the cells from the culture medium;
d) administering a solution comprising culture medium obtained from step c) to a site of tissue or organ damage.

51. The method according to claim 50, wherein said solution in step d) is injected to the site of tissue or organ damage.

52. The method according to claim 50, wherein said organ damage is ischemic damage in the heart.

53. The method according to claim 50, wherein said site of tissue or organ damage is myocardium.

54. The method according to claim 50, wherein said solution in step d) is administered topically to a site of tissue damage.

55. The method according to claim 50, wherein said tissue damage is a skin burn or wound.

56. The method according to claim 50, wherein one of the secreted growth factors in step b) is hepatocyte growth factor, HGF.

57. The method according to claim 50, wherein said solution in step d) comprises a purified fraction of the culture medium obtained from step c).

58. A pharmaceutical composition for use in treating tissue and/or organ damage in a patient, said composition comprising multicellular spheroids obtainable culturing fibroblast cells on a nonadhesive surface that induces the cells to adhere to each other so that multicellular spheroids are formed and triggers necrosis of the cells.

59. The composition according to claim 58, wherein said composition is a transplant or graft to be transplanted to a patient.

60. The composition according to claim 58, wherein said composition is an injectable solution.

61. The composition according to claim 58, wherein said composition is for topical administration.

62. The composition according to claim 58, wherein said fibroblasts are bone marrow stromal cells.

63. A pharmaceutical composition for use in treating tissue and/or organ damage in a patient, said composition comprising a solution obtainable by culturing fibroblast cells on a nonadhesive surface that induces the cells to adhere to each other so that multicellular spheroids are formed and triggers necrosis of the cells, incubating said multicellular spheroids under conditions that said fibroblast cells secrete growth factors to the culture medium, and separating the cells from the medium.

64. The composition according to claim 63, wherein said composition is an injectable solution.

65. The composition according to claim 63, wherein said composition is for topical administration.
